(19) United States
(12) Patent Application Publication
(10) Pub. No.: US 2014/0275183 A1
(43) Pub. Date: Sep. 18, 2014

(54) AGENT FOR REDUCING SIDE EFFECTS OF KINASE INHIBITOR

(71) Applicants: Ajinomoto Co., Inc., Chuo-ku (JP); Yamaguchi University, Yamaguchi-shi (JP)

(72) Inventors: Naoki YAMAMOTO, Ube-shi (JP); Isao SAKAIDA, Ube-shi (JP)

(73) Assignees: Ajinomoto Co., Inc., Chuo-ku (JP); Yamaguchi University, Yamaguchi-shi (JP)

(21) Appl. No.: 14/290,275

(22) Filed: May 29, 2014

Related U.S. Application Data
(63) Continuation of application No. PCT/JP2012/081213, filed on Nov. 30, 2012.

(30) Foreign Application Priority Data
Dec. 2, 2011 (JP) 2011-264687

(51) Int. Cl. A61K 31/198; A61K 31/44

(52) U.S. Cl. CPC A61K 31/198; A61K 31/44
USPC 514/350; 514/561

(57) ABSTRACT

The present invention relates to an agent for reducing the side effects of kinase inhibitors, the agent containing at least one branched-chain amino acid selected from among isoleucine, leucine and valine or a salt thereof as an active ingredient, and also relates to an anticancer medicine containing at least one branched-chain amino acid selected from among isoleucine, leucine and valine or a salt thereof, and a kinase inhibitor.

### CROSS-REFERENCE TO RELATED APPLICATIONS

The present application is a continuation of International Application No. PCT/JP2012/081213, filed Nov. 30, 2012, which claims the benefits of priority to Japanese Patent Application No. 2011-264687, filed Dec. 2, 2011. The entire contents of these applications are incorporated herein by reference.

### TECHNICAL FIELD

The present invention relates to an agent for reducing the side effects of a kinase inhibitor, an anticancer medicine which combines a branched-chain amino acid and a kinase inhibitor, and a medical kit or the like containing the same.

### BACKGROUND ART

Inhibitors of the various kinases, which participate in the proliferation of tumor cells and angiogenesis and the like, hold much promise as anticancer drugs. For example, the kinase inhibitor sorafenib, which exhibits in vivo inhibitory activity against a wide variety of kinases, is already in use clinically as an anticancer drug, particularly against hepatocellular carcinoma and renal cell carcinoma.

On the other hand, many anticancer drugs have significant side effects, and depending on the symptoms or severity of those side effects, administration of the anticancer drug may have to be abandoned in some cases. For example, ingestion of sorafenib causes hand-foot syndrome or bleeding from various tissues with relatively high frequency. Hand-foot syndrome is the generic name given to a side effect that occurs in the skin and nails of the hands and feet upon ingestion of the anticancer drug. The initial symptoms include sensory abnormalities such as numbness or tingling in the hands and feet, or the type of pain associated with burning, and in severe cases, causes intense pain that makes everyday life impossible. In this manner, the quality of life (QOL) of the patient is severely impaired, and therefore when hand-foot syndrome occurs, the administered dose of sorafenib must be reduced, or the administration of sorafenib must be temporarily or permanently halted. As a result, a method that reduces side effects while enabling the anticancer effect to manifest satisfactorily is keenly sought.

In some cases, using a combination of a plurality of anticancer drugs having different modes of action can yield a similar anticancer effect to that obtained by administering the agents individually, but with lower doses of the drugs. For example, Patent Document 1 discloses the use, as an anticancer drug, of a combination of a VEGFR tyrosine kinase inhibitor or a pharmaceutically acceptable salt thereof and a mTOR-selective kinase inhibitor or a pharmaceutically acceptable salt thereof, and lists sorafenib as one example of the VEGFR tyrosine kinase inhibitor.

On the other hand, it is known that branched-chain amino acids (BCAA) are effective against liver disease such as liver cancer. For example, Non-Patent Document 1 discloses that in a mouse model for non-alcoholic steatohepatitis (NASH), BCAA not only improved insulin resistance, but was also able to inhibit fatty change in the liver, and that administration of BCAA is therefore useful in preventing non-alcoholic fatty liver disease and inhibiting progression to NASH. Further, Patent Document 2 discloses that administration of BCAA can inhibit the development and progression of liver cancer. Moreover, the document also discloses that joint administration of BCAA and interferon enhances the anti-hepatitis C viral activity of interferon, and can also reduce the side effects of interferon such as fever.

In addition, Patent Document 4 discloses that a composition containing at least two or more types of essential amino acids, in which leucine in free or salt form is present in an amount of at least approximately 10 to 35% by weight based on the combined weight of all amino acids, is effective in improving cachexia (a state of severe malnutrition and negative nitrogen balance characterized by anorexia, namely loss of appetite, or severe weight loss and muscle atrophy) or weight loss caused by cancer.

### DOCUMENTS OF RELATED ART

### Patent Documents

Patent Document 1: Published Japanese Translation No. 2011-512395 of PCT
Patent Document 2: International Patent Publication No. 2004/058243
Patent Document 3: International Patent Publication No. 2007/013677
Patent Document 4: Published Japanese Translation No. 2006-503105 of PCT

### Non-Patent Documents

Non-Patent Document 1: Nemoto et al., “Investigation of the Utility of Branched-Chain Amino Acids (BCAA) in a Mouse Model for Non-Alcoholic Steatohepatitis (NASH)”, The Japanese Journal of Gastroenterology, Vol. 105, Extra Edition, A832 (2008).

### SUMMARY OF THE INVENTION

### Problems to be Solved by the Invention

The aforementioned Patent Documents 1 to 4 and the Non-Patent Document 1 make absolutely no mention of a method that is capable of directly reducing the side effects of kinase inhibitors such as sorafenib.

The present invention has an object of providing an agent for reducing the side effects of kinase inhibitors such as sorafenib.

### Means to Solve the Problems

As a result of intensive investigation aimed at achieving the above object, the inventors of the present invention discovered that by jointly taking the kinase inhibitor sorafenib, which acts as an anticancer drug, and at least one branched-chain amino acid selected from among isoleucine, leucine and valine or a salt thereof, the side effects of sorafenib could be reduced, and they were therefore able to complete the present invention.

In other words, the present invention provides side effect-reducing agents (1) to (9), anticancer medicines (10) to (17), and medical kits (18) and (19), all of which are described below.

(1) An agent for reducing a side effect of a kinase inhibitor, the agent containing at least one branched-chain amino acid selected from among isoleucine, leucine and valine or a salt thereof as an active ingredient.

(2) The agent for reducing a side effect according to (1), wherein the kinase inhibitor is a kinase inhibitor of at least one kinase selected from the group consisting of KIT, FLT-3, RET, VEGFR-1, VEGFR-2, VEGFR-3, PDGFR-13 and Raf.

(3) The agent for reducing a side effect according to (1) above, wherein the kinase inhibitor is 4-[4-[[4-chloro-3-(trifluoromethyl)phenyl]carbamoylamino]phenoxy]-N-methylpyridine-2-carboxamide, 4-[4-({[4-chloro-3-(trifluoromethyl)phenyl]carbamoyl}amino)-3-fluorophenoxy]-N-methylpyridine-2-carboxamide, N-(2-chloro-4-((6,7-dimethoxy-4-quinazolinyl)oxy)phenyl)-N′-propylurea, N-[3-[5-(2-aminopyrimidin-4-yl)-2-tert-butyl-1,3-thiazol-4-yl]-2-fluorophenyl]-2,6-difluorobenzenesulfonamide, 1-[1-[(2-aminopyridin-4-yl)methyl]indol-4-yl]-3-(5-bromo-2-methoxyphenyl)urea hydrochloride, N-[4-(3-amino-1H-indazol-4-yl)phenyl]-N′-(2-fluoro-5-methylphenyl)urea, 1-[2-chloro-4-(6,7-dimethoxyquinolin-4-yl)oxyphenyl]-3-(5-methyl-1,2-oxazol-3-yl)urea, 1-N′-[3-fluoro-4-[6-methoxy-7-(3-morpholin-4-ylpropoxy)quinolin-4-yl]oxyphenyl]-1-N-(4-fluorophenyl)cyclopropane-1,1-dicarboxamide, N-[[3-fluoro-4-[2-(1-methylimidazol-4-yl)thieno[3,2-b]pyridin-7-yl]oxyphenyl]carbamothioyl]-2-phenylacetamide, 1-N′-[2-fluoro-4-[2-[[4-(4-methylpiperazin-1-yl)piperidine-1-carbonyl]amino]pyridin-4-yl]oxyphenyl]-1-N-(4-fluorophenyl)cyclopropane-1,1-dicarboxamide, 4-[3-chloro-4-[[(cyclopropylamino)carbonyl]amino]phenoxy]-7-methoxy-monomethanesulfonate, 1-N′-(4-fluorophenyl)-1-N-[4-[[2-(2-morpholin-4-ylethylcarbamoyl)-1H-pyrrolo[2,3-b]pyridin-4-yl]oxy]phenyl]cyclopropane-1,1-dicarboxamide, 1-methyl-5-[2-[5-(trifluoromethyl)-1H-imidazol-2-yl]pyridin-4-yl]oxy-N-[4-(trifluoromethyl)phenyl]benzimidazol-2-amine, 6-[7-[(1-aminocyclopropyl)methoxy]-6-methoxyquinolin-4-yl]oxy-N-methylnaphthalene-1-carboxamide, or 2-dimethyl-6-[7-(2-morpholin-4-ylethoxy)quinolin-4-yl]oxy-1-benzofuran-3-carboxamide.

(4) The agent for reducing a side effect according to (1) above, wherein the kinase inhibitor is 4-[4-[[4-chloro-3-(trifluoromethyl)phenyl]carbamoylamino]phenoxy]-N-methylpyridine-2-carboxamide.

(5) The agent for reducing a side effect according to any one of (1) to (4) above, composed of the three branched-chain amino acids isoleucine, leucine and valine.

(6) The agent for reducing a side effect according to any one of (1) to (5) above, wherein the weight ratio between isoleucine, leucine and valine is 1:(1.5 to 2.5):(0.8 to 1.7).

(7) The agent for reducing a side effect according to any one of (1) to (6) above, wherein the side effect is at least one side effect selected from the group consisting of hand-foot syndrome and bleeding.

(8) The agent for reducing a side effect according to any one of (1) to (7) above, wherein the agent is administered to a liver cancer patient.

(9) The agent for reducing a side effect according to any one of (1) to (8) above, wherein the agent is administered to a patient having a liver stiffness stage of Child's classification A.

(10) An anticancer medicine, containing a combination of:

at least one branched-chain amino acid selected from among isoleucine, leucine and valine or a salt thereof, and

a kinase inhibitor.

(11) The anticancer medicine according to (10) above, wherein the kinase inhibitor is 4-[4-[[4-chloro-3-(trifluoromethyl)phenyl]carbamoylamino]phenoxy]-N-methylpyridine-2-carboxamide, 4-[4-({[4-chloro-3-(trifluoromethyl)phenyl]carbamoyl}amino)-3-fluorophenoxy]-N-methylpyridine-2-carboxamide, N-(2-chloro-4-((6,7-dimethoxy-4-quinazolinyl)oxy)phenyl)-N′-propylurea, N-[3-[5-(2-aminopyrimidin-4-yl)-2-tert-butyl-1,3-thiazol-4-yl]-2-fluorophenyl]-2,6-difluorobenzenesulfonamide, 1-[1-[(2-aminopyridin-4-yl)methyl]indol-4-yl]-3-(5-bromo-2-methoxyphenyl)urea hydrochloride, N-[4-(3-amino-1H-indazol-4-yl)phenyl]-N′-(2-fluoro-5-methylphenyl)urea, 1-[2-chloro-4-(6,7-dimethoxyquinolin-4-yl)oxyphenyl]-3-(5-methyl-1,2-oxazol-3-yl)urea, 1-N′-[3-fluoro-4-[6-methoxy-7-(3-morpholin-4-ylpropoxy)quinolin-4-yl]oxyphenyl]-1-N-(4-fluorophenyl)cyclopropane-1,1-dicarboxamide, N-[[3-fluoro-4-[2-(1-methylimidazol-4-yl)thieno[3,2-b]pyridin-7-yl]oxyphenyl]carbamothioyl]-2-phenylacetamide, 1-N′-[2-fluoro-4-[2-[[4-(4-methylpiperazin-1-yl)piperidine-1-carbonyl]amino]pyridin-4-yl]oxyphenyl]-1-N-(4-fluorophenyl)cyclopropane-1,1-dicarboxamide, 4-[3-chloro-4-[[(cyclopropylamino)carbonyl]amino]phenoxy]-7-methoxy-monomethanesulfonate, 1-N′-(4-fluorophenyl)-1-N-[4-[[2-(2-morpholin-4-ylethylcarbamoyl)-1H-pyrrolo[2,3-b]pyridin-4-yl]oxy]phenyl]cyclopropane-1,1-dicarboxamide, 1-methyl-5-[2-[5-(trifluoromethyl)-1H-imidazol-2-yl]pyridin-4-yl]oxy-N-[4-(trifluoromethyl)phenyl]benzimidazol-2-amine, 6-[7-[(1-aminocyclopropyl)methoxy]-6-methoxyquinolin-4-yl]oxy-N-methylnaphthalene-1-carboxamide, or 2-dimethyl-6-[7-(2-morpholin-4-ylethoxy)quinolin-4-yl]oxy-1-benzofuran-3-carboxamide.

(12) The anticancer medicine according to (10) above, wherein the kinase inhibitor is 4-[4-[[4-chloro-3-(trifluoromethyl)phenyl]carbamoylamino]phenoxy]-N-methylpyridine-2-carboxamide.

(13) The anticancer medicine according to any one of (9) to (11) above, composed of the three branched-chain amino acids isoleucine, leucine and valine.

(14) The anticancer medicine according to any one of (10) to (13) above, wherein the weight ratio between isoleucine, leucine and valine is 1:(1.5 to 2.5):(0.8 to 1.7).

(15) The anticancer medicine according to any one of (10) to (14) above, wherein the anticancer medicine is administered to a liver cancer patient.

(16) The anticancer medicine according to any one of (10) to (15) above, wherein the anticancer medicine is administered to a patient having a liver stiffness stage of Child's classification A.

(17) The anticancer medicine according to any one of (10) to (15) above, wherein the branched-chain amino acid or the salt thereof is taken together with the kinase inhibitor.

(18) A medical kit containing:

at least one branched-chain amino acid selected from among isoleucine, leucine and valine or a salt thereof, and

a kinase inhibitor.

(19) The medical kit according to 18 above, also containing documentation disclosing that the branched-chain amino acid or the salt thereof can be used, or should be used, for reducing the side effects of the kinase inhibitor.

### Effects of the Invention

The agent for reducing side effects according to the present invention can effectively reduce the side effects of kinase inhibitors such as sorafenib. As a result, by using the agent for reducing side effects of the present invention, or an anticancer medicine or medical kit containing this agent, in the treatment of diseases to which the various kinase inhibitors are applied, and particularly in the treatment of cancer, the quality of life (QOL) of the patient can be improved, and a superior therapeutic effect can be obtained.

### BRIEF DESCRIPTION OF THE DRAWINGS

FIG. 1 is a diagram illustrating the changes over time in the survival rate of each group in an Example 1.

FIG. 2 is a diagram illustrating the cell proliferation inhibitory effect of each group in an Example 2.

FIG. 3 is a diagram illustrating the antitumor action enhancement effect of each group in an Example 3.

### BEST MODE FOR CARRYING OUT THE INVENTION

### Branched-Chain Amino Acid

The isoleucine, leucine and valine which function as an active ingredient (branched-chain amino acid) of the present invention may each be used in the form of the L-isomer, the D-isomer or the DL-isomer, but the L-isomer or DL-isomer is preferable, and the L-isomer is the most desirable. Further, the isoleucine, leucine and valine may be used not only in free form, but also in the form of salts. Examples of these salts include acid addition salts and salts formed with bases, but the selection of a pharmaceutically acceptable salt of isoleucine, leucine or valine is preferable. Examples of acids which can be added to isoleucine, leucine and valine to form pharmaceutically acceptable salts include inorganic salts such as hydrogen chloride, hydrogen bromide, sulfuric acid and phosphoric salt, and organic acids such as acetic acid, lactic acid, citric acid, tartaric acid, maleic acid, fumaric acid and monomethyl sulfuric acid. Examples of pharmaceutically acceptable salts of isoleucine, leucine and valine with bases include salts formed with inorganic bases such as ammonia or the hydroxides or carbonates of metals such as sodium, potassium and calcium, and salts formed with organic bases such as ethylenediamine, propylenediamine, ethanolamine, monoalkylethanolamines, dialkylethanolamines, diethanolamine and triethanolamine.

### <Kinase Inhibitor>

In the present invention and the present description, the term “kinase inhibitor” includes inhibitors of cell surface kinases such as KIT, FLT-3, RET, VEGFR-1, VEGFR-2, VEGFR-3 and PDGFR-13, and inhibitors of intracellular kinases such as Raf.

Specific examples of the kinase inhibitors for which the side effects are reduced by using the side effect-reducing agent of the present invention include the compounds listed below.
4-[4-[[4-chloro-3-(trifluoromethyl)phenyl]carbamoylamino]phenoxy]-N-methylpyridine-2-carboxamide (sorafenib),
4-[4-({[4-chloro-3-(trifluoromethyl)phenyl]carbamoyl}amino)-3-fluorophenoxy]-N-methylpyridine-2-carboxamide,
N-(2-chloro-4-((6,7-dimethoxy-4-quinazolinyl)oxy)phenyl)-N′-propylurea,
N-[3-[5-(2-aminopyrimidin-4-yl)-2-tert-butyl-1,3-thiazol-4-yl]-2-fluorophenyl]-2,6-difluorobenzenesulfonamide,
1-[1-[(2-aminopyridin-4-yl)methyl]indol-4-yl]-3-(5-bromo-2-methoxyphenyl)urea hydrochloride,
N-[4-(3-amino-1H-indazol-4-yl)phenyl]-N′-(2-fluoro-5-methylphenyl)urea,
1-[2-chloro-4-(6,7-dimethoxyquinolin-4-yl)oxyphenyl]-3-(5-methyl-1,2-oxazol-3-yl)urea,
1-N-[3-fluoro-4-[6-methoxy-7-(3-morpholin-4-ylpropoxy)quinolin-4-yl]oxyphenyl]-1-N-(4-fluorophenyl)cyclopropane-1,1-dicarboxamide,
N-[[3-fluoro-4-[2-(1-methylimidazol-4-yl)thieno[3,2-b]pyridin-7-yl]oxyphenyl]carbamothioyl]-2-phenylacetamide,
1-N′-[2-fluoro-4-[2-[[4-(4-methylpiperazin-1-yl)piperidine-1-carbonyl]amino]pyridin-4-yl]oxyphenyl]-1-N-(4-fluorophenyl)cyclopropane-1,1-dicarboxamide,
4-[3-chloro-4-[[(cyclopropylamino)carbonyl]amino]phenoxy]-7-methoxy-monomethanesulfonate,
1-N′-(4-fluorophenyl)-1-N-[4-[[2-(2-morpholin-4-ylethylcarbamoyl)-1H-pyrrolo[2,3-b]pyridin-4-yl]oxy]phenyl]cyclopropanc-1,1-dicarboxamidc,
1-methyl-5-[2-[5-(trifluoromethyl)-1H-imidazol-2-yl]pyridin-4-yl]oxy-N-[4-(trifluoromethyl)phenyl]benzimidazol-2-amine,
6-[7-[(1-aminocyclopropyl)methoxy]-6-methoxyquinolin-4-yl]oxy-N-methylnaphthalene-1-carboxamide, and
2-dimethyl-6-[7-(2-morpholin-4-ylethoxy)quinolin-4-yl]oxy-1-benzofuran-3-carboxamide.

In the present invention, the kinase inhibitor is preferably sorafenib.

The kinase inhibitor may be in the form of a pharmaceutically acceptable salt. Examples of acids which can form a pharmaceutically acceptable salt with the kinase inhibitor include the same acids as those mentioned above in relation to the branched-chain amino acid.

### <Agent for Reducing Side Effects>

The agent for reducing side effects according to the present invention contains at least one branched-chain amino acid selected from among isoleucine, leucine and valine or a salt thereof as an active ingredient, and has the property of reducing the side effects of kinase inhibitors. The agent for reducing side effects according to the present invention may contain any one or more branched-chain amino acids selected from among isoleucine, leucine and valine, and preferably contains all three of isoleucine, leucine and valine.

The isoleucine, leucine and valine which represent the active ingredients of the present invention may be included in formulations either individually or in arbitrary combinations, or all three active ingredients may be included within a single formulation. When administered as separate formulations, the administration route and administration form may be the same or different for each formulation, and the timing of the administration of each formulation may also be either simultaneous or different. These decisions may be made as appropriate in accordance with the variety and effects of the drug being used in combination with the branched-chain amino acids.

When the three types of branched-chain amino acid are included in the agent for reducing side effects according to the present invention, the blend ratio (weight ratio) between the isoleucine, leucine and valine is preferably 1:(1 to 3):(0.5 to 2.0), more preferably 1:(1.5 to 2.5):(0.8 to 1.7), still more preferably 1:(1.5 to 2.5):(0.8 to 1.5), still more preferably 1:(1.9 to 2.2):(1.1 to 1.3), and particularly preferably 1:2:1.2.

In the present invention, the “weight ratio” indicates the weight of each component within a formulation. For example, when isoleucine, leucine and valine are all included in a single formulation, the weight ratio indicates the amount of each active ingredient, whereas when the active ingredients are included across a plurality of formulations, either individually or in arbitrary combinations, the weight ratio describes the ratio of the weight of each active ingredient in each formulation.

Further, in the present invention, the actual dose ratio describes the ratio between the active ingredients per administration subject (namely, each patient) for a single dose or a daily dose. For example, when each of the active ingredients of isoleucine, leucine and valine are included in a single formulation, and that formulation is administered to a subject, the weight ratio is equal to the dose ratio. When the active ingredients are administered across a plurality of formulations, either individually or in arbitrary combinations, the ratio of the total of each active ingredient across each of the formulations administered in a single dose or across one day corresponds with the weight ratio.

Isoleucine, leucine and valine are already widely used in the fields of pharmaceuticals and foodstuffs, and their safety is well established. For example, the acute toxicity (LD₅₀) in a pharmaceutical composition of the present invention containing these amino acids in a ratio of 1:2:1.2 was more than 10 g/kg in the case of oral administration to mice.

The agent for reducing side effects according to the present invention can be formulated using normal methods to form solid formulation such as powders, granules, capsules, tablets or chewable tablets, liquid formulations such as solutions and syrups, or other formulations such as injections and sprays. These formulations can be administered by any appropriate administration method including oral administration, injection or local administration.

The agent for reducing side effects according to the present invention can be formulated by blending the branched-chain amino acids of the active ingredient with appropriate pharmaceutically acceptable carriers as required, including excipients, binders, lubricants, solvents, disintegrants, solubilizers, suspending agents, emulsifiers, isotonizing agents, stabilizers, pain relievers, preservatives, antioxidants, correctives and colorants.

Examples of the excipients include organic excipients, including sugars such as lactose, glucose and D-mannitol, starches, and celluloses such as crystalline cellulose, and inorganic excipients such as calcium carbonate and kaolin. Examples of the binders include α-starch, gelatin, gum arabic, methyl cellulose, carboxymethyl cellulose, sodium carboxymethylcellulose, crystalline cellulose, D-mannitol, trehalose, hydroxypropyl cellulose, hydroxypropylmethyl cellulose, polyvinylpyrrolidone and polyvinyl alcohol. Examples of the lubricants include stearic acid, fatty acid salts such as stearates, talc and silicates. Examples of the solvents include purified water and physiological saline solution. Examples of the disintegrants include low-substitution degree hydroxypropyl cellulose and chemically modified celluloses and starches. Examples of the solubilizers include polyethylene glycol, polypropylene glycol, trehalose, benzyl benzoate, ethanol, sodium carbonate, sodium citrate, sodium salicylate and sodium acetate. Examples of the suspending agents and emulsifiers include sodium laurate, gum arabic, gelatin, lecithin, glycerol monostearate, polyvinyl alcohol, polyvinylpyrrolidone, celluloses such as sodium carboxymethyl cellulose, polysorbates and polyoxyethylene hardened castor oil. Examples of the isotonizing agents include sodium chloride, potassium chloride, sugars, glycerol and urea. Examples of the stabilizers include polyethylene glycol, dextran sulfate sodium, and other amino acids. Examples of the pain relievers include glucose, calcium gluconate and procaine hydrochloride. Examples of the preservatives include paraoxybenzoate esters, chlorobutanol, benzyl alcohol, phenethyl alcohol, dehydroacetic acid and sorbic acid. Examples of the antioxidants include sulfite salts and ascorbic acid. Examples of the correctives include the types of sweeteners and flavorings typically used in the fields of pharmaceuticals and foodstuffs. Examples of the colorants include the types of coloring materials typically used in the fields of pharmaceuticals and foodstuffs.

By taking the agent for reducing side effects of the present invention, the side effects of kinase inhibitors are reduced. The agent for reducing side effects of the present invention is preferably taken to reduce the side effects of at least one of the group of kinase inhibitors specified above, and among this group of kinase inhibitors, the agent for reducing side effects is preferably taken to reduce the side effects of a kinase inhibitor taken as an anticancer drug, and more preferably taken to reduce the side effects of sorafenib. Because branched-chain amino acids themselves have an effect in inhibiting the development and progression of liver cancer (see Patent Document 2), the agent for reducing side effects according to the present invention is most preferably taken to reduce the side effects of sorafenib that has been administered as an anticancer drug for treating liver cancer.

Examples of the side effects that are reduced by the agent for reducing side effects according to the present invention include hand-foot syndrome, exfoliative dermatitis, mucocutaneous ocular syndrome (Stevens-Johnson syndrome), erythema multiforme, bleeding (including gastrointestinal bleeding, respiratory tract bleeding, cerebral hemorrhaging, oral bleeding, nose bleeding, nail bed bleeding, hematoma and tumor hemorrhaging), acute hepatitis, liver function disorder or jaundice, liver failure, hepatic encephalopathy, as well as liver function disorders, jaundice, liver failure and hepatic encephalopathy accompanied by elevated AST (GOT) or ALT (GPT), and acute lung injury, interstitial pneumonia, hypertensive crisis, reversible posterior leukoencephalopathy syndrome, myocardial ischemia or myocardial infarction, congestive heart failure, gastrointestinal perforation, gastrointestinal ulceration, hemorrhagic enteritis, ischemic enteritis, leukopenia, neutropenia, lymphopenia, thrombopenia, anemia, pancreatitis, renal failure, shock, anaphylactic reaction and rhabdomyolysis. Other side effects include hypersensitivity (including cutaneous reaction and hives), elevated INR, prolonged prothrombin time, rash, hair loss, pruritus, erythema, dry skin, acne, skin scale, eczema, redness, depression, sensory peripheral neuropathy, tinnitus, dizziness, joint pain, muscular pain, hoarseness, rhinorrhea, hypertension, QT prolongation, diarrhea, nausea, vomiting, elevated amylase, elevated lipase, constipation, mouth stomatitis (including mouth dryness and tongue pain), indigestion, dysphagia, anorexia, gastroesophageal reflux disease, gastritis, elevated AST (GOT), elevated ALT (GPT), elevated bilirubin, elevated Al—P, gallbladder inflammation, cholangitis, elevated LDH, fatigue, pain (including oral pain, stomach pain, osteocopic pain, headache and cancer pain), hypophosphatemia, asthenia, fever, influenza-like symptoms, weight loss, erectile dysfunction, folliculitis, infection, gynecomastia, hypothyroidism, hyponatremia, dehydration, dysgeusia, hyperthyroidism, radiation recall reaction, hyperkalemia and edema.

In particular, the agent for reducing side effects according to the present invention is preferably taken with the aim of reducing one or more side effects selected from among cutaneous symptoms such as hand-foot syndrome, exfoliative dermatitis, mucocutaneous ocular syndrome (Stevens-Johnson syndrome), erythema multiforme, rash, hair loss, pruritus, erythema, dry skin, acne, skin scale, eczema and redness, and bleeding (including gastrointestinal bleeding, respiratory tract bleeding, cerebral hemorrhaging, oral bleeding, nose bleeding, nail bed bleeding, hematoma and tumor hemorrhaging).

By taking the agent for reducing side effects according to the present invention in combination with a kinase inhibitor such as sorafenib, the side effects of the kinase inhibitor can be reduced. As a result, by taking the agent for reducing side effects according to the present invention, the QOL of a patient taking the kinase inhibitor can be improved. Further, in terms of the dose of the kinase inhibitor, even patients who, due to the severity of side effects, could conventionally only be administered with a small dose of the kinase inhibitor, are able to be more safely administered with a dose sufficient to achieve the desired drug efficacy. Administration of the agent for reducing side effects to patients having a liver stiffness stage of Child's classification A is particularly preferable. A patient with a Child's classification A describes a patient having a bilirubin level lower than 2.0 mg/dl and an albumin level higher than 3.5 g/dl, and is a patient having a comparatively good nutritional status with no symptoms of ascites or encephalopathy.

For example, when sorafenib is used as an anticancer drug, by taking the sorafenib in combination with the agent for reducing side effects of the present invention, side effects such as hand-foot syndrome and bleeding can be reduced dramatically, meaning that even for patients for whom the severity of these side effects would normally have resulted in the abandonment or temporary suspension of treatment, the sorafenib can be administered without any excessive loss in QOL. Furthermore, because of the lightness of the side effects, the form of administration can be selected more freely, allowing the dose to be increased or the administration period to be lengthened, and therefore the range of possible treatment plans can be broadened.

Besides the branched-chain amino acid or salt thereof, the agent for reducing side effects according to the present invention may also contain other medical components as active ingredients. Examples of these other medical components include anticancer drugs other than the aforementioned kinase inhibitors, and nutrients and the like such as vitamins.

### <Anticancer Medicine>

The anticancer medicine of the present invention contains a combination of at least one branched-chain amino acid selected from among isoleucine, leucine and valine or a salt thereof (namely, the agent for reducing side effects according to the present invention), and a kinase inhibitor, and is administered to a subject for the purpose of treating cancer. The anticancer medicine of the present invention may be a medicine in which the agent for reducing side effects according to the present invention and the kinase inhibitor are included in a single formulation, or a medicine in which the two components are combined as individual formulations.

The anticancer medicine of the present invention includes not only medicines used for treating cancer, but also medicines that inhibit the development or progression of cancer.

The kinase inhibitor can be formulated as any of a variety of formulations using conventional methods and pharmaceutically acceptable carriers and additives. Examples of the types of formulations and suitable pharmaceutically acceptable carriers, which may be used as required depending on the formulation, are the same as those listed above in relation to the agent for reducing side effects according to the present invention.

Any of the various commercially available kinase inhibitor formulations can be used as the kinase inhibitor. For example, sorafenib is available commercially as tablets of the tosylate salt under the brand name Nexavar (a registered trademark) (manufactured by Bayer Pharma AG).

There are no particular limitations on the patients used as treatment subjects for the anticancer medicine of the present invention, provided they are humans or animals suffering from cancer (malignant tumor). For example, the anticancer medicine may be used for treating primary cancer or metastatic cancer. Furthermore, the cancer may be early-stage cancer or advanced cancer. For example, when the kinase inhibitor is sorafenib, the anticancer medicine is preferably administered for the treatment of renal cell carcinoma and hepatocellular carcinoma, and is most preferably administered to patients having unresectable hepatocellular carcinoma and having a liver stiffness stage of Child's classification A.

### <Administration Method, Dose>

In the agent for reducing side effects and the anticancer medicine according to the present invention, the administered dose (intake) of the branched-chain amino acids varies depending on the condition and age of the patient and the administration method and the like, but is typically equivalent to a dose, per person per day, of 0.5 to 30.0 g of isoleucine, 1.0 to 60.0 g of leucine, and 0.5 to 30.0 g of valine. In the case of a typical adult, the dose per person per day is preferably 2.0 to 10.0 g of isoleucine, 3.0 to 20.0 g of leucine, and 2.0 to 10.0 g of valine, and more preferably 2.5 to 3.5 g of isoleucine, 5.0 to 7.0 g of leucine, and 3.0 to 4.0 g of valine. When the agent includes all three of isoleucine, leucine and valine, the administered dose per adult per day, calculated as the combined total of all three types of branched-chain amino acid, is typically approximately 2.0 to 50.0 g, which if required, may be divided into 1 to 6 administered portions, and preferably 1 to 3 portions.

The amount of branched-chain amino acids in a single administered dose is typically approximately 0.5 to 50.0 g, preferably approximately 1.0 to 20.0 g, and more preferably approximately 2.0 to 6.0 g.

There are no particular limitations on the administered dose, administration period, administration schedule or administration route or the like of the kinase inhibitor, provided that the efficacy of the kinase inhibitor manifests satisfactorily. For example, the dose for each of the kinase inhibitors specified above, regardless of the administration route, may be within a range from 0.01 to 200 mg/kg (bodyweight) per day. When the kinase inhibitor is sorafenib, an adult is preferably orally administered with 400 mg of sorafenib per dose, either every second day, or 1 or 2 times per day.

In the present invention, the branched-chain amino acids and the kinase inhibitor may be administered as separate formulations, using either the same or different forms of administration. Alternatively, they may be administered as a combination drug containing both the branched-chain amino acids and the kinase inhibitor.

When the branched-chain amino acids and the kinase inhibitor are separate formulations, there are no particular limitations on the form of administration used, provided that the branched-chain amino acids and the kinase inhibitor both exist in vivo at the same time, and for example, the branched-chain amino acids may be administered at the same time as the kinase inhibitor, or administered either before or after the kinase inhibitor. The administration method and doses used in the case of joint administration may be selected appropriately in accordance with the type of drug being used and the effects of the drug.

When the branched-chain amino acids and the kinase inhibitor are administered with a time difference therebetween, the time difference may vary depending on the administered active ingredients, the dosage form and the administration method, and for example in the case where the branched-chain amino acids are administered first, the kinase inhibitor is typically administered within a period of 5 minutes to 14 days, and more preferably within a period of 10 minutes to 7 days, from the administration of the branched-chain amino acids. When the kinase inhibitor is administered first, the branched-chain amino acids are typically administered within a period of 5 minutes to 120 hours, and more preferably within a period of 10 minutes to 80 hours, from the administration of the kinase inhibitor.

When calculating the dose (intake) of the active ingredient branched-chain amino acids used in the present invention, the numerical range described above is determined on the basis of the amount of the active ingredient within the medicine used for the purpose of reducing the side effects of the kinase inhibitor, and therefore if branched-chain amino acids are also taken or administered for other purposes, such as within the normal daily diet or for the treatment of a different disease, these amounts need not be included within the above calculations. For example, the amount of branched-chain amino acids consumed per day as part of a normal diet need not be subtracted from the daily administration dose of the active ingredient in the present invention.

### <Medical Kit>

The one or more types of branched-chain amino acid selected from among isoleucine, leucine and valine or a salt thereof (namely, the agent for reducing side effects according to the present invention) may be combined with an effective amount of a kinase inhibitor formulation in the form of individual formulations, and these formulations may then be packaged together to form a medical kit.

The medical kit preferably also contains documentation disclosing that the agent for reducing side effects according to the present invention can be used, or should be used, for reducing the side effects of the kinase inhibitor. This documentation may be added to the kit in the form of a document printed on paper or the like, may be printed directly onto the container containing the agent for reducing side effects according to the present invention, or may be printed directly onto the box or plastic bag or the like containing both the agent for reducing side effects according to the present invention and the kinase inhibitor. Instead of printing directly to the container or box or the like, a printed seal may also be affixed.

### EXAMPLES

The present invention is described below in further detail using a series of examples and the like, but the present invention is in no way limited by these examples.

### Example 1

Using CDAA (choline-deficient L-amino acid defined) diet-induced liver cancer model rats, the effects of the combined intake of sorafenib and branched-chain amino acids (BCAA) were investigated. The CDAA diet used a commercially available choline-deficient diet (#518753: Choline deficient and iron supplemented L-amino acid defined rat diet, manufactured by Dyets Inc.).

Six-week old Fischer 344 rats were divided into a CDAA diet intake group, a sorafenib only intake group, and a BCAA combined intake group (N=15 for each group), and the test diet was provided freely. For the CDAA diet intake group, only the CDAA diet was supplied as the test diet. For the sorafenib only intake group, a CDAA diet mixed with an amount of sorafenib tosylate tablets (Nexavar (a registered trademark) tablets 200 mg, manufactured by Bayer Pharma AG) sufficient to provide an intake of sorafenib of 16 mg/kg/day (800 mg/50 kg/day) (hereafter referred to as the “CDAA/sorafenib diet”) was supplied. For the BCAA combined intake group, a diet prepared by mixing the CDAA/sorafenib diet supplied to the sorafenib only intake group with 2.5% by mass of a BCAA formulation (Livact (a registered trademark) granules, manufactured by Ajinomoto Co., Inc.) (hereafter referred to as the “CDAA/sorafenib/BCAA diet”) was supplied. The weight ratio between isoleucine, leucine and valine in the BCAA formulation was 1:2:1.2 (isoleucine:0.952 g, leucine: 1.904 g, and valine: 1.144 g).

Each individual was autopsied either at the time of death or after 56 weeks from the start of the test (from supply of the test diet), and the effect of treatment continuation was investigated on the basis of the hepatic fibrosis inhibitory effect, the hepatocarcinogenesis rate and the survival rate.

Further, the presence or absence of hepatic fibrosis was determined on the basis of the result of Azan staining of a liver tissue section, and the result of measuring the concentration of HYP (hydroxyproline) within the liver. The presence or absence of hepatocarcinogenesis was evaluated by immunostaining a tissue section of the liver with an antibody to the precancerous marker GST-P (glutathione S-transferase placental form), and inspecting the tissue section for the presence of staining.

The changes over time in the survival rates for each group are shown in FIG. 1. In the CDAA diet intake group and the BCAA combined intake group, all of the rats were alive 20 weeks after starting the test, whereas in the sorafenib only intake group, all the individuals died 16 to 20 weeks after starting the test, displaying cutaneous symptoms or whole body bleeding, indicating a significant increase in the death rate. In other words, in the BCAA combined intake group, the survival rate improved dramatically compared with the sorafenib only intake group. Further, compared with the CDAA diet intake group, the number of rats in which hepatic fibrosis or precancerous lesions were observed was fewer in the sorafenib only intake group and the BCAA combined intake group, confirming that the onset of liver cancer was inhibited in these two groups. Furthermore, in the sorafenib only intake group, redness was noted not only on the four legs, but also on the trunk and the skin, and a tendency for bleeding to occur in the gastrointestinal tract, including the anal region, was also observed. Moreover, a loss of appetite due to gastrostenosis was also noted. In contrast, in the BCAA combined intake group, the symptoms of redness, bleeding and loss of appetite were all improved significantly.

Based on these results, it was clear that in a choline-deficient L-amino acid defined diet-induced hepatic fibrosis and liver cancer rat model, by jointly administering sorafenib and BCAA, a hepatic fibrosis inhibitory effect and a liver precancerous lesion inhibitory effect were obtained, and the survival rate and level of side effects were improved significantly compared with the case of administering only sorafenib, enabling continued treatment to be performed safely.

### Example 2

### Cell Proliferation Inhibitory Effect

Each of the wells of a 96 well plate was inoculated with 6×10³ cell/well of Huh? cells, and on the following day, the medium was exchanged with an LC medium (containing 10% FBS), sorafenib of concentration 0, 1, 2, 4 or 8 μM containing 2 mM of BCAA was added, and incubation was performed for 48 hours. Samples containing no added BCAA were also incubated for 48 hours as controls.

After 48 hours, the medium was removed, 4% paraformaldehyde was added to each well and fixation was performed for 15 minutes. Following removal of the paraformaldehyde, a 5% Hoechst solution was added to each well, and left to stand for one minute. The 5% Hoechst solution was then removed, and 200 μl of a PBS solution was added to each well.

The plate was analyzed for cell count using an array scan, and the cell count in each group was calculated as a % of cell proliferation relative to a value of 100 for the cell count of the control (LC 10%). The results are shown in FIG. 2.

As is evident from FIG. 2, in the sorafenib+BCAA groups, and particularly in the cases where combined stimulation with BCAA was used in the 2, 4 and 8 μM stimulation with sorafenib, a significant cell proliferation inhibitory effect was observed compared with the case of stimulation with only sorafenib.

### Example 3

### Antitumor Action Enhancement Effect

Huh7 cells were implanted subcutaneously in BALB/c nude mice in an amount of 1×10⁷ cell/mouse.

After one week, the mice were split into groups based on tumor diameter, and the groups were subjected to 2 weeks of 5 mg/kg sorafenib oral administration±food supply containing 3% BCAA (with the sorafenib administered 5 days/week).

In the third week of administration, the sorafenib dose was increased from 5 mg/kg to 30 mg/kg, and oral administration was continued for 5 days. During this time, supply of the feed containing 3% BCAA was continued.

Three weeks after the start of administration, the tumor diameter of each group was measured, and the average value+SE for the tumor volume was calculated. The tumor volume was calculated as tumor major axis/2×(minor axis)².

The results are shown in FIG. 3.

As is evident from the results of FIG. 3, in the sorafenib+BCAA group, the degree of shrinkage in the tumor volume was large compared with the control group, and a tendency for an enhanced antitumor effect was observed as a result of combined administration of the two components.

In the sorafenib only group and the BCAA only group, the degree of shrinkage in the tumor volume was unchanged from that of the control group.

### INDUSTRIAL APPLICABILITY

The agent for reducing side effects according to the present invention can effectively reduce the side effects of kinase inhibitors such as sorafenib. As a result, the agent for reducing side effects of the present invention, and an anticancer medicine and medical kit containing this agent, are ideal for the medical treatment of diseases to which the various kinase inhibitors are applied, and particularly for the treatment of cancers that are treated using sorafenib.

1. An agent for reducing a side effect of a kinase inhibitor, the agent comprising at least one branched-chain amino acid selected from among isoleucine, leucine and valine or a salt thereof as an active ingredient.

2. The agent for reducing a side effect according to claim 1, wherein the kinase inhibitor is 4-[4-[[4-chloro-3-(trifluoromethyl)phenyl]carbamoylamino]phenoxy]-N-methylpyridine-2-carboxamide, 4-[4-({[4-chloro-3-(trifluoromethyl)phenyl]carbamoyl}amino)-3-fluorophenoxy]-N-methylpyridine-2-carboxamide, N-(2-chloro-4-((6,7-dimethoxy-4-quinazolinyl)oxy)phenyl)-N′-propylurea, N-[3-[5-(2-aminopyrimidin-4-yl)-2-tert-butyl-1,3-thiazol-4-yl]-2-fluorophenyl]-2,6-difluorobenzenesulfonamide, 1-[1-[(2-aminopyridin-4-yl)methyl]indol-4-yl]-3-(5-bromo-2-methoxyphenyl)urea hydrochloride, N-[4-(3-amino-1H-indazol-4-yl)phenyl]-N′-(2-fluoro-5-methylphenyl)urea, 1-[2-chloro-4-(6,7-dimethoxyquinolin-4-yl)oxyphenyl]-3-(5-methyl-1,2-oxazol-3-yl)urea, 1-N′-[3-fluoro-4-[6-methoxy-7-(3-morpholin-4-ylpropoxy)quinolin-4-yl]oxyphenyl]-1-N-(4-fluorophenyl)cyclopropane-1,1-dicarboxamide, N-[[3-fluoro-4-[2-(1-methylimidazol-4-yl)thieno[3,2-b]pyridin-7-yl]oxyphenyl]carbamothioyl]-2-phenylacetamide, 1-N′-[2-fluoro-4-[2-[[4-(4-methylpiperazin-1-yl)piperidine-1-carbonyl]amino]pyridin-4-yl]oxyphenyl]-1-N-(4-fluorophenyl)cyclopropane-1,1-dicarboxamide, 4-[3-chloro-4-[[(cyclopropylamino)carbonyl]amino]phenoxy]-7-methoxy-monomethanesulfonate, 1-N′-(4-fluorophenyl)-1-N-[4-[[2-(2-morpholin-4-ylethylcarbamoyl)-1H-pyrrolo[2,3-b]pyridin-4-yl]oxy]phenyl]cyclopropane-1,1-dicarboxamide, 1-methyl-5-[2-[5-(trifluoromethyl)-1H-imidazol-2-yl]pyridin-4-yl]oxy-N-[4-(trifluoromethyl)phenyl]benzimidazol-2-amine, 6-[7-[(1-aminocyclopropyl)methoxy]-6-methoxyquinolin-4-yl]oxy-N-methylnaphthalene-1-carboxamide, or 2-dimethyl-6-[7-(2-morpholin-4-ylethoxy)quinolin-4-yl]oxy-1-benzofuran-3-carboxamide.

3. The agent for reducing a side effect according to claim 1, wherein the kinase inhibitor is 4-[4-[[4-chloro-3-(trifluoromethyl)phenyl]carbamoylamino]phenoxy]-N-methylpyridine-2-carboxamide.

4. The agent for reducing a side effect according to claim 1, comprising three branched-chain amino acids isoleucine, leucine and valine.

5. The agent for reducing a side effect according to claim 1, wherein a weight ratio between isoleucine, leucine and valine is 1:(1.5 to 2.5):(0.8 to 1.7).

6. The agent for reducing a side effect according to claim 1, wherein the side effect is at least one side effect selected from the group consisting of hand-foot syndrome and bleeding.

7. The agent for reducing a side effect according to claim 1, wherein the agent is administered to a liver cancer patient.

8. The agent for reducing a side effect according to claim 1, wherein the agent is administered to a patient having a liver stiffness stage of Child's classification A.

9. An anticancer medicine, comprising a combination of:
at least one branched-chain amino acid selected from among isoleucine, leucine and valine or a salt thereof, and
a kinase inhibitor.

10. The anticancer medicine according to claim 9, wherein the kinase inhibitor is 4-[4-[[4-chloro-3-(trifluoromethyl)phenyl]carbamoylamino]phenoxy]-N-methylpyridine-2-carboxamide, 4-[4-({[4-chloro-3-(trifluoromethyl)phenyl]carbamoyl}amino)-3-fluorophenoxy]-N-methylpyridine-2-carboxamide, N-(2-chloro-4-((6,7-dimethoxy-4-quinazolinyl)oxy)phenyl)-N′-propylurea, N-[3-[5-(2-aminopyrimidin-4-yl)-2-tert-butyl-1,3-thiazol-4-yl]-2-fluorophenyl]-2,6-difluorobenzenesulfonamide, 1-[1-[(2-aminopyridin-4-yl)methyl]indol-4-yl]-3-(5-bromo-2-methoxyphenyl)urea hydrochloride, N-[4-(3-amino-1H-indazol-4-yl)phenyl]-N′-(2-fluoro-5-methylphenyl)urea, 1-[2-chloro-4-(6,7-dimethoxyquinolin-4-yl)oxyphenyl]-3-(5-methyl-1,2-oxazol-3-yl)urea, 1-N′-[3-fluoro-4-[6-methoxy-7-(3-morpholin-4-ylpropoxy)quinolin-4-yl]oxyphenyl]-1-N-(4-fluorophenyl)cyclopropane-1,1-dicarboxamide, N-[[3-fluoro-4-[2-(1-methylimidazol-4-yl)thieno[3,2-b]pyridin-7-yl]oxyphenyl]carbamothioyl]-2-phenylacetamide, 1-N′-[2-fluoro-4-[2-[[4-(4-methylpiperazin-1-yl)piperidine-1-carbonyl]amino]pyridin-4-yl]oxyphenyl]-1-N-(4-fluorophenyl)cyclopropane-1,1-dicarboxamide, 4-[3-chloro-4-[[(cyclopropylamino)carbonyl]amino]phenoxy]-7-methoxy-monomethanesulfonate, 1-N′-(4-fluorophenyl)-1-N-[4-[[2-(2-morpholin-4-ylethylcarbamoyl)-1H-pyrrolo[2,3-b]pyridin-4-yl]oxy]phenyl]cyclopropane-1,1-dicarboxamide, 1-methyl-5-[2-[5-(trifluoromethyl)-1H-imidazol-2-yl]pyridin-4-yl]oxy-N-[4-(trifluoromethyl)phenyl]benzimidazol-2-amine, 6-[7-[(1-aminocyclopropyl)methoxy]-6-methoxyquinolin-4-yl]oxy-N-methylnaphthalene-1-carboxamide, or 2-dimethyl-6-[7-(2-morpholin-4-ylethoxy)quinolin-4-yl]oxy-1-benzofuran-3-carboxamide.

11. The anticancer medicine according to claim 9, wherein the kinase inhibitor is 4-[4-[[4-chloro-3-(trifluoromethyl)phenyl]carbamoylamino]phenoxy]-N-methylpyridine-2-carboxamide.

12. The anticancer medicine according to claim 9, comprising three branched-chain amino acids isoleucine, leucine and valine.

13. The anticancer medicine according to claim 9, wherein a weight ratio between isoleucine, leucine and valine is 1:(1.5 to 2.5):(0.8 to 1.7).

14. The anticancer medicine according to claim 9, wherein the anticancer medicine is administered to a liver cancer patient.

15. The anticancer medicine according to claim 9, wherein the anticancer medicine is administered to a patient having a liver stiffness stage of Child's classification A.

16. The anticancer medicine according to claim 9, wherein the branched-chain amino acid or the salt thereof is taken together with the kinase inhibitor.

17. A medical kit comprising:
at least one branched-chain amino acid selected from among isoleucine, leucine and valine or a salt thereof, and
a kinase inhibitor.

18. The medical kit according to claim 17, further comprising documentation disclosing that the branched-chain amino acid or the salt thereof can be used, or should be used, for reducing side effects of the kinase inhibitor.
